(19) United States
(12) Patent Application Publication
(10) Pub. No.: US 2004/0031067 A1
(43) Pub. Date: Feb. 12, 2004

(54) Regulation of human skin healing

(76) Inventors: Meenhard Herlyn, Wynnewood, PA (US); Carola Berking, Munich (DE); Kapaettu Satyamoorthy, Santhekatte (IN); Omaida Velazquez, Cherry Hill, NJ (US)

Correspondence Address: HOWSON AND HOWSON, ONE SPRING HOUSE CORPORATION CENTER, BOX 457, 321 NORRISTOWN ROAD, SPRING HOUSE, PA, 19477, US

(21) Appl. No.: 10/398,980

(22) PCT Filed: Oct. 11, 2001
(86) PCT No.: PCT/US01/31555

(51) Int. Cl. C12N 5/08; A01K 67/00; C12N 15/63; C12N 15/87; C12Q 1/68

(52) U.S. Cl. 800/9; 435/6; 435/455

(57) ABSTRACT

The invention relates to new compositions and methods for preparing vascularized dermal reconstructs, vascularized skin grafts, and for enhancing vascularization in situ in response to a variety of medical conditions.

This application claims the priority of U.S. provisional application 60/239,123, filed Oct. 11, 2000, which is hereby incorporated by reference in its entirety.

### TECHNICAL FIELD OF THE INVENTION

The invention relates to the regulation of human skin healing. In particular, the invention relates to the identification and use of substances which promote vascularization of injured or diseased skin and other tissues.

### BACKGROUND OF THE INVENTION

The human skin consists of a multilayered epidermis with keratinocytes, melanocytes and immune cells and a thick, well-vascularized dermis. By contrast, the mouse skin consists of only 3 to 4 keratinocyte layers in the epidermis and a thin dermis. It is thus difficult to apply results on tissue reorganization in wound healing, cancer, or inflammation in mice to humans.

A human skin/SCID mouse chimera model in which full-thickness human skin is grafted to mice was developed to address this issue. (Yan, 1993). In the model, the human epidermis remains unchanged, whereas the dermis may be infiltrated by mouse vessels and a relatively mild inflammatory cell infiltrate is seen (Yan, 1993). The human skin graft model has been used successfully for studies in wound healing (Sylvester, 2000, Liechety, 1999), inflammation (Oka, 2000), carcinogenesis (Juhasz, 1993), and tumor growth and metastasis (Sauter, 1999).

Injection of recombinant proteins into skin or their application to wounds enables studies on their potential for repair and remodeling. However, recombinant proteins are rapidly degraded. The expression of a gene, in situ, is an attractive alternative to direct delivery of proteins, for studies on gene function. Adenoviral vectors readily infect all human skin cells and have attracted wide attention for efficient gene transduction (Setoguchei, 1994).

The use of in vitro skin reconstructs is another approach to providing treatment for medical conditions which require repair of the skin. Early in vitro models for the study of angiogenesis were limited by the use of animal-derived endothelial cells, the absence of properly polarized networks with true lumens, very high serum supplementation (20%), the use of tumor promoters for endothelial cell induction, and/or the use of various purified extracellular matrix components as part of three-dimensional scaffold such as fibrin, fibrinogen, or the multimolecular matrix, Matrigel (Montesano 1983, 1985, 1986, 1993, Kubota 1988, Ingber 1989, Jackson 1991, Chalupowicz 1995). In an effort to mimic or induce physiologic three-dimensional endothelial cell differentiation, investigators have usually mixed the endothelial cells (usually of human umbilical vein origin) within the various types of matrices, prior to the polymerization of the gels, bypassing or aiding the process of matrix invasion and migration (Yang 1999, Gerritsen 2000, Schechner 2000). Others have started from fragments of harvested arterial or venous segments (usually of animal origin), and embedded them into various types of gels using high serum supplementation and the exogenous addition of growth factors (Hoying 1996, Nicosia 1990, Hoying 1996 a,b). A recent in vitro human angiogenesis model employed complex extracellular multi-molecular structural matrices to mimic a basement membrane, with high serum supplementation (Black 1998) and/or the addition of exogenous growth factors (Foda 1996, Yang 1999, Gerritsen 2000). In addition, recent studies of human angiogenesis have been performed on umbilical vein endothelial cells (Black 1998, Gerritsen, Yang 1999, 2000, Schechner 2000). However, those models do not closely mimic the biologic behavior of human capillary networks which are composed of human microvascular endothelial cells.

Wounds, diabetes, and cancers are examples of medical conditions in which vascularization plays an important role. Improved vascularization is needed in wound repair and diabetes, while limiting vascularization will help control cancerous growth.

A need exists for identification of test substances which are therapeutically useful. A need exists for methods of treating disease states which are characterized by impaired vascularization, such as wounds and diabetes. A need exists for substances that can control melanoma.

A need also exists for in vitro reconstructs made of normal non-immortalized human cells which allows angiogenesis of human epithelial tissues such as skin.

### BRIEF DESCRIPTION OF THE DRAWINGS

FIG. 1 shows the vascularization response of a skin graft to the expression of various growth factors. Vessel formation is shown on the ordinate axis on a relative scale (1+ to 4+; left of axis) based on the number of vessels per mm² (130 to >210, right of axis) assessed on a microscope high power field of histological sections. Proliferation of pericytes is presented on the abscissa in the same manner using a relative (1+ to 4+, above axis) and absolute scale (420 to >840 pericytes per mm²). The vectors encoding the polypeptides in the box did not produce a response different from the lacZ control vectors.

FIG. 2 shows the connective tissue response of a skin graft to the expression of various growth factors. Proliferation of fibroblastic cells is shown on the abscissa on a relative scale (1+ to 4+; above axis) based on the number of fibroblastic cells per mm² (800 to >2600) assessed on a microscope high power field of histological section. Induction of matrix is presented on the abscissa using a relative scale (1+ to 4+, above axis). Vectors in the box were not different from the lacZ control vectors.

FIG. 3 shows the epidermal reaction of a skin graft to the expression of various growth factors. Papillomatosis is shown on the ordinate on a relative scale (1+ to 4+; left of axis) based on the number of papillae per mm of epidermis (7 to >56, right of axis) assessed on a microscope high power field of a histological section. Epidermal hyperplasia is presented on the abscissa in the same manner using a relative scale (1+ to 4+, above axis) and average epidermal thickness (0.08 to >0.64 mm). Vectors in the box were not different from the lacZ control vectors.

FIG. 4 shows a schematic representation of angiogenesis in a dermal reconstruct. HMVEC are plated on collagen type I coated plates and grown as a monolayer to 80% confluency, in complete medium. Then, a first layer of acellular collagen is allowed to polymerize overlying the endothelial cell monolayer. Subsequently, a second layer of collagen with embedded fibroblasts is also allowed to polymerize overlying the first layer. The gels are bathed in medium during the assembly of the reconstruct.

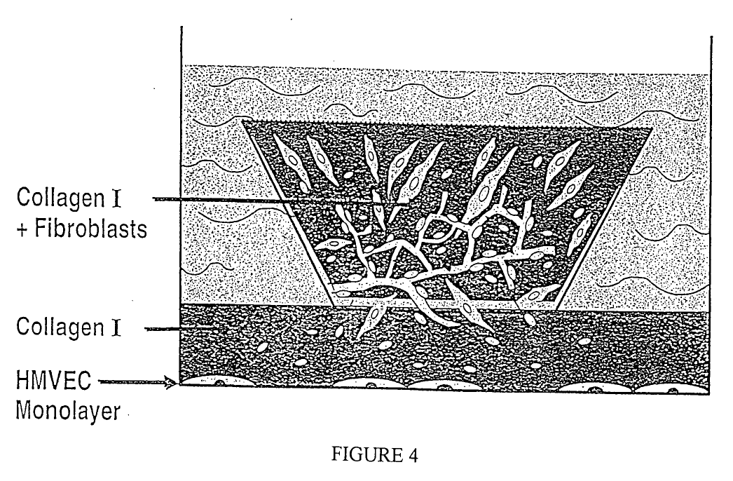

FIG. 5 shows the induction of branching of microcapillaries by fibroblasts expressing different growth factors in a dermal reconstruct. Branching was counted after fibroblasts were transduced with growth factor genes using adenoviral vectors. One day after transduction, fibroblasts were embedded in collagen type I and layed over endothelial cells, which then migrated into the constricting collagen and differentiated to form tubes. Results are expressed as the number of branching capillary-like structures 5 days after embedding of fibroblasts in collagen and seeding over endothelial cells.

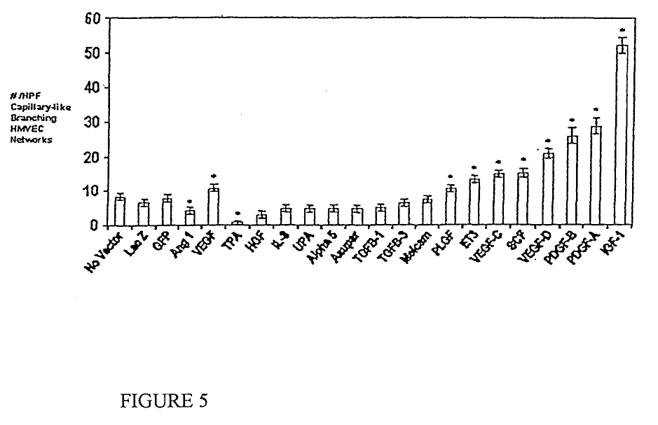

### SUMMARY OF THE INVENTION

It is an object of the invention to provide a method of treating medical conditions requiring vascularization of tissue. This and other objects of the invention are provided by one or more of the embodiments described below.

One embodiment of the invention is a method of producing an autologous vascularized human skin graft in a patient. The method comprises: injecting intradermally into an autologous skin graft a vector comprising a polynucleotide encoding tPA, MEL-CAM, uPA, SCF, bFGF, IGF-1, or ET-3, whereby vasculature develops into said skin graft.

Another embodiment of the invention provides a skin graft system comprising an immuno-deficient mouse with a human skin graft, wherein the graft has been intradermally injected with a vector comprising a polynucleotide encoding tPA, MEL-CAM, uPA, SCF, bFGF, IGF-1 or ET-3. The mouse can be a SCID mouse.

Another embodiment of the invention provides a method of treating a mammalian patient suffering from a medical condition which can be improved by vascularization of a tissue. The method comprises:

injecting into the mammalian patient a vector comprising a polynucleotide encoding a substance selected from the group consisting of tPA, MEL-CAM, uPA, SCF, bFGF, IGF-1, and ET-3.

Yet another embodiment of the invention provides a microvascularized dermal reconstruct. The reconstruct is made of a matrix comprising:

a first layer comprising collagen 1 and second layer comprising fibroblast cells and collagen 1, wherein the matrix is overlaid on a monolayer of human endothelial cells, wherein the first layer is adjacent to said monolayer of human endothelial cells.

Still another embodiment of the invention provides a method for producing an in vitro vascularized dermal reconstruct, comprising: growing separate monolayer cultures of human endothelial cells, fibroblast cells, and smooth muscle cells; transducing at least one of the cultures of cells with a vector comprising a polynucleotide encoding VEGF, Ang 1, Ang 2, tPA, uPA, MEL-CAM, SCF, PDGF-A, VEGF-D, TGF-b1, VEGF-C, PDGF-B, bFGF, IGF-1, or ET-3; overlaying on said human endothelial cells a first layer comprising collagen 1; overlaying on the first layer a second layer comprising collagen 1, the fibroblast cells and the smooth muscle cells, whereby a dermal reconstruct is formed; and growing the reconstruct in a culture to achieve vascularization.

A further embodiment of the invention provides a method of treatment of a medical condition, comprising: placing at the site of a wound, a burn, ischemic tissue, or in the limb of a diabetic patient, a vascularized reconstruct made by the methods of the invention.

The invention thus provides substances which can be administered to a patient which suffers from a medical condition whose treatment requires an improvement of vascularization

### DETAILED DESCRIPTION OF THE INVENTION

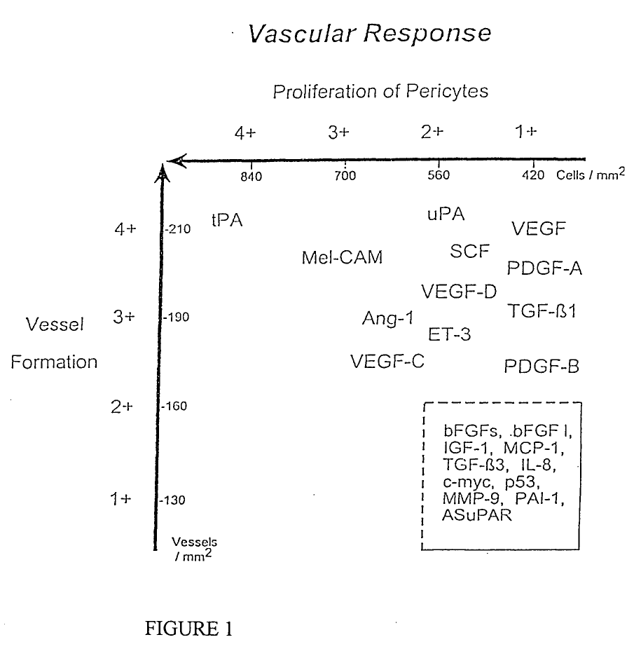

One aspect of the present invention is based on an observation that certain substances when injected into human skin affect the development of a proper graft by altering connective tissue development, epidermis formation and, most importantly, induction of vascularization. A total of 48 polypeptides were tested in a human skin patch grafted onto a mouse. They are described in Tables 1 and 2. A ranking of those substances in relation to their effects on pericyte proliferation and vessel formation (vascularization), matrix or stromia development and fibroblast proliferation (connective tissue development), and epidermal hyperplasia and papillomatosis (epidermis formation), respectively, is shown in FIGS. 1-3.

Most important and surprising was the high degree of vascularization induced by tPA, uPA, Mel-CAM, SCF, bFGF, IGF-1 and ET-3. In particular, tPA induced vascularization without causing significant immune inflammation. Citations below provide the actual nucleic acid sequences encoding the polypeptides discussed. Their contents are incorporated herein by reference. The numbers refer to GenBank Accession numbers.

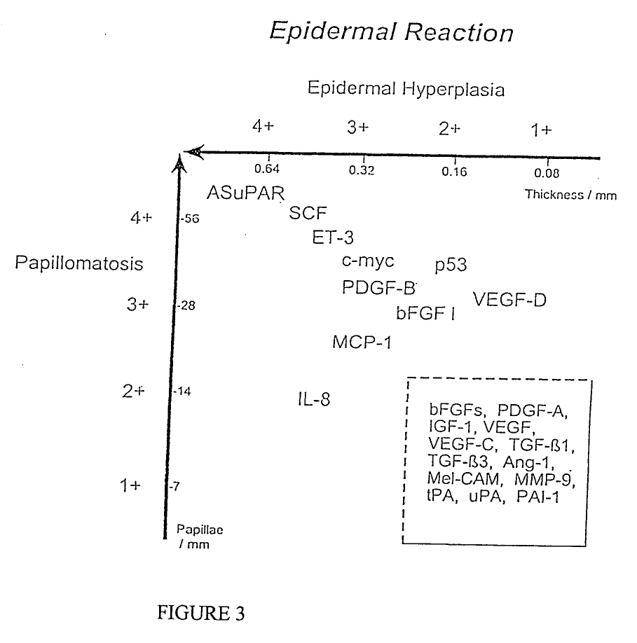

The data shown in FIG. 3 indicate that bFGF has only a background level of activity. However, repeated delivery (at least 3 times) of the virus engineered to express bFGFs demonstrated that bFGFs can significantly induce vascularization. It appears that gene products that are slowly released from cells may require repeated injections.

The genes encoding those polypeptides were introduced into the genome of replication deficient adenoviral vectors by standard genetic engineering technology. Viral stocks were propagated in tissue culture by known methods in the art. For testing the effect of those polypeptides, the virus was delivered intradermally into a grafted patch. However, similar results would be obtained if the patch is injected with the vector prior to grafting. The intradermal injection of the viral vectors in 100 μl buffer resulted in dissemination of the virus throughout the grafts. The human skin graft was 1.5 to 2 cm².

The grafting of skin is a well known procedure. It is done for treatment of medical conditions, treatment of burn victims, and for research purposes. The grafting of human skin onto experimental animals, and in particular into immunodeficient mice such as SCID mice is known in the art.

In a preferred embodiment, one or more of the tPA, uPA, Mel-CAM, SCF, bFGF, IGF-1, or ET-3 polypeptides themselves can be injected onto a skin graft in a patient. Alternatively, one or more of these polypeptides can be delivered into a patient absent the need for a skin patch. In the later case, the injection is preferably at a site in the body requiring improved vascularization and development of a tissue such as skin. Examples of medical conditions which benefit from treatment with tPA, uPA, Mel-CAM, SCF, bFGF, IGF-1, or ET-3, or with skin grafts expressing those factors, include trauma wounds, wounds related to diabetes, foot ulcers, venous leg ulcers, and burns.

The polypeptides can be administered as purified substances, or, preferably, are delivered via a nucleic acid vector. For example, the vector can be a virus genome or a plasmid which is engineered to express at least one of the polypeptides. Delivery of the genes via a replication defective adenovirus or retrovirus is a preferred embodiment, although any suitable vector can be employed.

The inventors have discovered that skin grafts injected with polynucleotides which encode one or more of the tPA, uPA, Mel-CAM, SCF, bFGF, IGF-1, or ET-3 can recruit bone marrow derived stem cells into the graft. Such stem cells become resident in the graft and differentiate to form endothelial cells and fibroblasts, thereby contributing to the development of vascularized skin tissue and augmenting endothelial cells, fibroblasts and their progenitors which are endogenous to the graft. Bone marrow derived stem cells can optionally be implanted into a skin graft (e.g., by intradermal injection of a cell suspension) in order to promote tissue development and vascularization. The stem cells may be obtained from any source, but are preferably obtained from the recipient of the graft (i.e., from an autologous donor). Bone marrow derived stem cells can be prepared, for example, according to Pereira et al. (Proc. Natl. Acad. Sci. USA 92:4857 (1995)) or Prockop (Science 276:71 (1997). More generally, any stem cell or progenitor cell type, or combinations of such cells, can be used which give rise in the graft to endothelial cells or fibroblasts, and preferably to both endothelial cells and fibroblasts. Differentiation of stem cells to form fibroblasts and endothelial cells can be observed using cell type-specific molecular markers which are known in the art. For example, endothelial cells are CD31+ and CD146+, whereas fibroblasts express fibroblast marker 451.

In another embodiment, the skin graft of the invention can be used in a method to test agents for treatment of skin diseases, for example melanoma. The testing for a therapeutic agent is best done in a skin graft system in a model animal. For example a SCID mouse having a human skin graft containing melanoma cells or other cancer cells can be prepared. Melanoma cells or other cancer cells can be implanted into the graft, e.g., by intradermal injection, or a skin patch can be grafted from a diseased animal, including a human, such as one who has melanoma or another cancer. The graft is optimized and vascularized by injection of at least one substance from among tPA, uPA, Mel-CAM, SCF, bFGF, IGF-1, and ET-3. Test substances to be tested for melanoma treatment can be applied by any method. These include, but are not limited to, delivery in an ointment, orally, or by any form of injection. The substances can be purified or expressed from nucleic acids. Cancer cells, including melanoma cells, are well suited for identification of therapeutic agents using a skin graft system because of their propensity to uncontrolled cellular proliferation, increased cellular motility and invasiveness, and ability to induce new blood vessel formation, all of which can be observed in a skin graft using available techniques.

The inventors have discovered that a variety of polypeptides are more highly expressed by melanoma cells, and such polypeptides may contribute to the growth of melanoma cells. Many of these polypeptides are extracellular matrix proteins or growth factors which promote the expression of extracellular matrix proteins. Such polypeptides include TGF-β1, collagen type VI, collagen type XV, collagen type XVIII, tenascin, PAI-I, VEGF, CFR-1, PDGF receptor β, or HGF. Thus, one possible class of test substances that offer promise in treating melanoma are antisense oligonucleotides comprising 15 or more consecutive nucleotides of the complement of a polynucleotide sequence encoding TGF-β1, collagen type VI, collagen type XV, collagen type XVIII, tenascin, PAI-I, VEGF, CFR-1, PDGF receptor β, or HGF. Another possible class of test substances that offer promise in treating melanoma are antibodies that specifically bind TGF-β1, collagen type VI, collagen type XV, collagen type XVIII, tenascin, PAI-I, VEGF, CFR-1, PDGF receptor β, or HGF polypeptides. Antisense oligonucleotides and antibodies, including monoclonal antibodies, can be produced according to techniques which are well known in the art. Relatedly, the inventors have discovered that expression of E-cadherin and desmoglein-1 promote adhesion between keratinocytes and melanocytes, yet the expression of both proteins is reduced in melanoma. The above described method of identifying therapeutic drugs for treatment of melanoma can also be employed in conjunction with transduction of any of the cells in the skin graft with a polynucleotide that encodes either E-cadherin and/or desmoglein-1.

TABLE 1
Adenoviral vectors for over-expression or suppression of
expression of growth factors in human skin.

| Growth factors, | | |
|---|---|---|
| cytokines and antagonists* | Abbreviation | References** |
| Basic Fibroblast Growth Factor | bFGF s | (Nesbit, 1999) |
| (18 kDa) | | |
| Basic Fibroblast Growth Factor | bFGF 1 | (Nesbit, 1999) |
| (18, 22, 23, 24 kDa) | | |
| Platelet-Derived Growth Factor A | PDGF-A | (N.A.) |
| Platelet-Derived Growth Factor B | PDGF-B | (Liechty, 1999) |
| Insulin-Like Growth Factor-1 | IGF-1 | (N.A.) |
| Monocyte chemoattractant protein-1 | MCP-1 | (N.A.) |
| Interleukin-8 | IL-8 | (Oka, 2000) |
| Vascular Endothelial Cell Growth | VEGF | (N.A.) |
| Factor-121 | | |
| Vascular Endothelial Cell Growth | VEGF-C | (N.A.) |
| Factor-C | | |
| Vascular Endothelial Cell Growth | VEGF-D | (N.A.) |
| Factor-D | | |
| Pleiotrophin | PLEIO | (Satyamoorthy, |
| | | 2000) |
| Transforming Growth Factor beta-1 | TGF-β1 | (Lee, 1998) |
| Transforming Growth Factor beta-3 | TGF-β3 | (N.A.) |
| Hepatocyte Growth Factor | HGF | (Phaneuf, 2000) |
| Angiopoitin-1 | Ang-1 | (Thurston, 2000) |
| Angiopoitin-2 | Ang-2 | (N.A.) |
| Stem Cell Factor | SCF | (N.A.) |
| Endothelin-3 | ET-3 | (N.A.) |
| Keratinocyte Growth Factor (murine) | KGF | (N.A.) |
| Fibroblast Growth Factor-10 (murine) | FGF-10 | (N.A.) |
| Antisense bFGF | ASbFGF | (Leconte, 1998) |
| Antisense Pleio | ASPleio | (N.A.) |
| Antisense IL-8 | AS IL-8 | (N.A.) |
| Dominant-negative mutant IGF-1 | DNM IGF-1R | (Lee, 1996) |
| receptor | | |

*All genes were of human origin unless otherwise indicated.
**References refer to characterization of the adenoviral vector.
N.A..= unpublished

TABLE 2
Adenoviral vectors for over-expression of proteases, inhibitors,
adhesion receptors, oncogenes, tumor suppressor genes,
and miscellaneous human genes.

| | Abbreviation | Reference** |
|---|---|---|
| Enzymes, their inhibitors and | | |
| receptor antagonists* | | |
| Matrix metalloproteinase-9 | MMP-9 | (Baker, 1996) |
| Tissue inhibitor of matrix | TIMP-1 | (Baker, 1996) |
| metalloproteinase-1 | | |
| Urokinase-type plasminogen activator | uPA | (Nagai, 1999) |
| Antisense urokinase-type | ASuPAR | (Mohan, 1999) |
| plasminogen activator receptor | | |
| Tissue-type plasminogen activator | tPA | (Carmeliet, 1997) |
| Plasminogen activator inhibitor-1 | PAI-1 | (Carmeliet, 1997) |
| Fibroblast growth factor | IGF-BP | (N.A..) |
| binding protein | | |
| Microophthalmia-associated | MITF | (N.A.) |
| transcription factor | | |
| LacZ | LacZ | |
| Green fluorescence protein | GFP | (de Martin, 1997) |
| Adhesion receptors* | | |
| β3-Integrin | Beta-3 | (Hsu, 1998) |
| Melanoma cell adhesion molecule | Mel-CAM | (N.A.) |
| (MUC18, CD146, S-Endo-1) | | |
| E-cadherin | E-CAD | (Hsu, 2000) |
| N-cadherin | N-CAD | (Hsu, 2000) |
| α2 integrin | α2 | (N.A.) |
| α5 integrin | α5 | (N.A.) |
| Oncogenes and tumor | | |
| suppressor genes* | | |
| Human papillomavirus (HPV) type | E6 | (N.A.) |
| 16 E6 oncogene | | |
| Human papillomavirus (HPV) type | E7 | (N.A.) |
| 16 E7 oncogene | | |
| Cellular homologue of avian | c-myc | (N.A.) |
| myelocytomatosis virus oncogene | | |
| p16 tumor suppressor protein | p16 | (N.A.) |
| p21 tumor suppressor protein | p21 | (Prabhu, 1996) |
| p53 tumor suppressor protein | p53 | (Prabhu, 1996) |
| Antisense cyclin D1 | AscycD1 | (Sauter, 1999) |

*All genes were of human origin unless otherwise indicated.
**Reference for adenoviral vector characterization.
N.A. = unpublished

In another embodiment, a skin graft in a model animal is wounded and injected with one or more of tPA, uPA, Mel-CAM, SCF, bFGF, IGF-1, or ET-3, or one or more polynucleotides which encode one or more of tPA, uPA, Mel-CAM, SCF, bFGF, IGF-1, or ET-3. A substance to be tested for therapeutic effect on wounds is applied. When this procedure is performed in the presence of a test substance, the test substance is identified as a candidate for the treatment of wounds if the rate of wound healing or degree of wound closure is increased compared to the same parameter in the absence of the test substance.

Another aspect of the invention involves the preparation of an in vitro reconstruct that physiologically mimics the human skin in respect to endothelial cell invasion, migration, proliferation, maturation, and, most significantly, differentiation into three-dimensional interconnecting tubes with a polarized true lumen.

Another use of the invention is a method of inducing vascularization in a mammalian tissue. Under certain medical conditions, mammalian tissues would benefit from increased vascularization. Such medical conditions include the healing of wounds in trauma, surgical wounds, wounds resulting as a consequence of diabetes, venous leg ulcers, and burns. Ischemia resulting from coronary atherosclerosis or following myocardial infarction is another such condition. Tissues involved in such medical conditions and requiring or benefitting from increased vascularization can be helped by the introduction of tPA, MEL-CAM, uPA, SCF, bFGF, IGF-1, or ET-3, any one or more of which will promote vascularization. Thus, for example, the tissue at the site of a wound, venous leg ulcer, burn, or an ischemic condition can be injected with a vector comprising a polynucleotide which encodes one or more of tPA, MEL-CAM, uPA, SCF, bFGF, IGF-1, or ET-3 to increase vascularization in the tissue. In some embodiments, bone marrow derived stem cells are also injected into the tissue to promote healing by the development of fibroblasts and endothelial cells, which further promote vascularization.

The invention has been further demonstrated by making a reconstruct comprising a human microvascular endothelial cell (HMVEC) layer which, unlike other known models, was not mixed within a supporting matrix or seeded over it. Instead, the HMVEC were grown as a monolayer and placed underneath the supporting matrix. The supporting matrix contains human dermal fibroblasts and collagen. In a preferred embodiment, the matrix also contains smooth muscle cells. The fibroblasts, smooth muscle cells, and endothelial cells are grown separately prior to assembly. The endothelial cells can be from any organ. Preferably, they are from an organ of the patient who will be the recipient of the reconstruct. The fibroblasts can also be from any source, but preferably they are from the same organ as the endothelial cells. The smooth muscle cells are preferably from the same patient as the endothelial cells. They are preferably isolated from the patient from a peripheral blood vessel biopsy. Optionally, any of these cells can be transfected to express a telomerase gene. The telomerase gene is known to prolong the life span of a non-transformed mammalian cell. In, some embodiments, bone marrow derived stem cells or other stem cells or progenitor cells which can produce fibroblasts and/or endothelial cells, as described above, are also added to the reconstruct.

Growth of the various cell types can be in any of a number of well known media. For example, the endothelial cells and the smooth muscle cells can be grown in either M199 (Sigma, St. Louis) or MCDB131 (Sigma, St. Louis) and the fibroblasts can be grown in DMEM+10% fetal bovine serum (Sigma, St. Louis), under standard and well known conditions.

After growth, any of the cells can be transduced by recombinant vectors described below. The transduction can be performed on cells in a medium-free condition by any method known in the art, for example, infection, liposome delivery, or electroporation. The vectors can be any replicating nucleic acid, for example a virus or a plasmid. Examples of suitable viruses are adenovirus, baculovirus, and retrovirus. In a preferred embodiment, a replication-deficient adenovirus is used. Methods of engineering the vectors for expression of desirable genes are well known in the art. Methods of selecting transformed cells are also well known.

It was observed by the inventors that a reconstruct where the cells were transduced to express VEGF, or VEGF and Ang 1 will develop a high density of incipient lumens. Reconstructs expressing VEGF, Ang 1, and Ang 2 achieved increased maturation of the lumen. In a preferred embodiment, the fibroblast is also transformed or transfected with a vector encoding at least one substance from among tPA, uPA, Mel-CAM, SCF, bFGF, IGF-1, and ET-3, to effect vascularization in the patient tissue where the reconstruct will be implanted.

It is not necessary that the genes encoding any of the above genes, VEGF, Ang 1, Ang 2, tPA, uPA, Mel-CAM, SCF, bFGF, IGF-1, or ET-3 be on one vector; multiple vectors can be used. Not all recipient cells need be transduced. Because the expressed substances are exported outside the host cell, not all cells need to be transduced by each and every vector. If more than one type of polypeptide is being expressed, then each polypeptide can be expressed in different cells or cell types, or in the same cells or cell types.

The reconstruct is built in layers. At the bottom is a layer of endothelium cells from which the growth medium has been removed. The matrix is built on top of the endothelial layer, as follows. The first layer is added on top of the endothelial cell layer. The first layer is an acellular layer of collagen type I, such as that available from Organogenesis (Canton, Mass.), which is allowed to polymerize. When polymerized, a second layer comprising collagen type I and fibroblasts is poured on top of the first layer and allowed to polymerize. The second layer preferably also contains smooth muscle cells. When the second layer has hardened, a supporting growth medium is added on top of the second layer. An example of an acceptable medium is modified MCDB131 to which 1-5% fetal bovine serum added. 1% fetal bovine serum is preferred.

The dermal reconstructs described above can be used as a wound cover or administered to tissue to stimulate wound healing, to replace wounded or missing tissue, or to stimulate vascularization and healing of adjacent tissues in conditions including wounds, ischemia, and diabetes.

It will be understood by one skilled in the art that the invention is not limited by the experimental details used to demonstrate the invention. The skilled artisan will recognize alternative embodiments and alternative materials. For example, a skin graft can be of any origin and the model animal is not limited to mouse. Similarly, the origin of endothelial cells and fibroblasts used by the invertors in demonstrating the principles of the invention do not limit the invention. Further, the skin reconstruct can be made to include an epidermis. See Meier et al, Am. J. Path. 156: 193-200 (2000), whose contents are incorporated herein by reference.

Moreover, the dermal reconstruct system described above can be modified to produce other artificial vascularized epithelial tissues. The basic features of the reconstruct, which are a monolayer of endothelial cells, a first layer of collagen type I, and a second layer comprising collagen type I and fibroblasts, can be modified by the addition of further cell types to the second layer to form reconstructs of non-dermal tissues. For example, the addition of smooth muscle cells to the second layer permits the development of larger vascular elements than capillaries. Similarly, other types of cells including a variety of epithelial cells can be added to the second layer to form a vascularized reconstruct of an epithelial tissue.

Expression Systems

The delivery of a therapeutic agent to a patient suffering from a medical condition such as diabetes, a burn, or a wound can be performed by any acceptable means, including delivery of a purified factor or a nucleic acid vector system which expresses the agent.

A number of viral-based expression systems can be used to deliver tPA, uPA, Mel-CAM, SCF, bFGF, IGF-1, or ET-3. For example, if an adenovirus is used as an expression vector, the genes encoding tPA, uPA, Mel-CAM, SCF, bFGF, IGF-1, or ET-3 can be ligated into an adenovirus at the late promoter and tripartite leader sequence. Insertion in a non-essential E1 or E3 region of the viral genome can be used to obtain a viable virus which is capable of expressing the tPA, uPA, Mel-CAM, SCF, bFGF, IGF-1, or ET-3 polypeptide in infected host cells (Logan & Shenk, Proc. Natl. Acad. Sci. 81, 3655□3659, 1984). If desired, transcription enhancers, such as the Rous sarcoma virus (RSV) enhancer, can be used.

Specific initiation signals also can be used to achieve more efficient translation. Such signals include the ATG initiation codon and adjacent sequences. The initiation codon should be in the correct reading frame to ensure translation of the entire coding region. Exogenous translational elements and initiation codons can be of various origins, both natural and synthetic. The efficiency of expression can be enhanced by the inclusion of enhancers which are appropriate for the particular cell system which is used (see Scharf et al., Results Probl. Cell Differ. 20, 125-162, 1994).

In another embodiment, the reagent is delivered using a liposome. A liposome comprises a lipid that is capable of delivery of a reagent to cells an animal, such as a human. A liposome useful in the present invention comprises a lipid composition that is capable of fusing with the plasma membrane of the targeted cell to deliver its contents to the cell. Suitable liposomes for use in the present invention include those liposomes standardly used in gene delivery methods known to those of skill in the art. Preferred liposomes include those having a polycationic lipid composition and/or liposomes having a cholesterol backbone conjugated to polyethylene glycol. Optionally, a liposome comprises a compound capable of targeting the liposome to a particular cell type, such as a cell-specific ligand exposed on the outer surface of the liposome which binds specifically to a receptor on the target cell.

Determination of a Therapeutically Effective Dose

The determination of a therapeutically effective dose is well within the capability of those skilled in the art. A therapeutically effective dose refers to that amount of active ingredient which increases or decreases activity relative to the activity which occurs in the absence of the therapeutically effective dose.

For any compound, the therapeutically effective dose can be estimated initially either in cell culture assays or in animal models, usually mice, rabbits, dogs, or pigs. The animal model also can be used to determine the appropriate concentration range and route of administration. Such information can then be used to determine useful doses and routes for administration in humans.

Therapeutic efficacy and toxicity, e.g., ED50 (the dose therapeutically effective in 50% of the population) and LD50 (the dose lethal to 50% of the population), can be determined by standard pharmaceutical procedures in cell cultures or experimental animals. The dose ratio of toxic to therapeutic effects is the therapeutic index, and it can be expressed as the ratio, LD50/ED50.

Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage form employed, sensitivity of the patient, and the route of administration.

The exact dosage will be determined by the practitioner. Dosage and administration are adjusted to provide sufficient levels of the active ingredient or to maintain the desired effect. Facts which can be taken into account include the severity of the disease state, general health of the subject, age, weight, and gender of the subject, diet, time and frequency of administration, drug combination(s), reaction sensitivities, and tolerance/response to therapy. Normal dosage amounts can vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

In any of the embodiments described above, any of the pharmaceutical compositions of the invention can be administered in combination with other appropriate therapeutic agents. Selection of the appropriate agents for use in combination therapy can be made by one of ordinary skill in the art, according to conventional pharmaceutical principles. The combination of therapeutic agents can act synergistically to effect the treatment or prevention of the various disorders described above. Using this approach, one may be able to achieve therapeutic efficacy with lower dosages of each agent, thus reducing the potential for adverse side effects.

Any of the therapeutic methods described above can be applied to any subject in need of such therapy, including, for example, mammals such as dogs, cats, cows, horses, rabbits, monkeys, and most preferably, humans.

All patents and patent applications cited in this disclosure are expressly incorporated herein by reference in their entirety. The above disclosure generally describes the present invention. A more complete understanding can be obtained by reference to the following specific examples which are provided for purposes of illustration only and are not intended to limit the scope of the invention.

### EXAMPLE 1

Adenovirus Preparation.

Replication deficient adenoviruses were constructed in which the genes were placed under a CMV promoter using standard protocols (Hsu, 2000). The plaque-purified viral vectors were propagated in human embryonic kidney 293 cells that complement the deleted viral E1 gene products. Virus was purified by cesium chloride centrifugation, dialyzed, and stored at −70° C. in 10 mM Tris/100 mM HCl, pH 8.1, with 10% glycerol. Viral titers were determined by plaque-forming assays.

### EXAMPLE 2

Human Skin Grafting.

C.B. 17 SCID mice between 4 and 6 weeks of age were used for grafting of human foreskin from newborns (Atillasoy, 1998). The 1.5 to 2 cm² full-thickness skin grafts were sutured into size-matched normal beds on the dorsal torso of the mice. Each mouse received a separate graft, which was allowed to heal for 4 to 6 weeks prior to injection of adenoviral vectors.

### EXAMPLE 3

Gene Transfer.

Human skin grafts were injected with 100 μl virus suspension containing 5×10⁸ plaque forming units (pfu) in phosphate-buffered saline. The viruses were injected intradermally with Hamilton gas-light syringes with 30-gauge needles (Reno, N.Y.). At least 2 to 3 grafts were injected with each vector. If significant tissue reorganization was seen after virus injection, experiments were repeated at least once, most often twice. The viruses were injected in groups of 5 including a lacZ control. Intradermal injection was confirmed by the presence of an epidermal wheel. The area of injection was marked with indelible ink. Three days following inspection, mice wee sacrificed and the grafts were harvested for histological analyses.

### EXAMPLE 4

Tissue Processing and Histological Evaluation.

The human skin grafts were dissected free from surrounding murine tissue and bisected perpendicular to the long axis of the graft. The grafts were fixed overnight in 10% neutral buffered formalin at 4° C. and parafin-embedded for histological assessment. The other half of the graft was cryopreserved in 20% sucrose overnight at 4° C.

For histological assessment, replicate serial sections (5 μm) from each of the formalin-fixed and paraffin-embedded grafts were stained with hematoxylin and cosin. The rate of acute inflammatory response, vascular response, connective tissue and epidermal reaction to the adenovirus-mediated transgene of each graft was assessed independently by 2 observers. Sections were first assessed for uniformity of staining at low power (×100). Individual counts for inflammatory cells including granulocytes, lymphocytes and monocytic cells, vessels, pericytes, and fibroblastic cells were then performed on a high power field (HPF, ×400). For microscope independent expression, counts were quantified as the number of structures/mm² (1 HPF=0.0496 mm²). Each count was performed independently on four representative HPFs by the two investigators. The induction of collagen was measured using Image-Pro®Plus analyzing software (Media Cybernetics, L.p., Silver Spring, Md.) on Masson trichome stained slides. With the help of this image analyzing software, the collagen specific color densitometric measurements were converted to optical density. Epidermal hyperplasia was evaluated by determining the number of papillae/mm of epidermal surface and measuring epidermal thickness. Changes in skin were deemed negative if observers agreed that they were below a threshold that was set for each of the evaluation criteria.

### EXAMPLE 5

β-Galactosidase Histochemistry.

Cryosections were fixed in 0.5% glutaraldehyde for 10 min, followed by rinsing twice in 1 mM MgCl₂/phosphate-buffered saline solution (PBS) for 10 min each. Slides were then incubated for 1 h in the dark at 37° C. in β-galactosidase incubation solution containing 1 mM MgCl2, 20×KC solution in PBS at pH 7.4, and β-galactosidase (5-bromo-4-chloro-3-indolyl β-galactopyranoside) at a final concentration of 1 mg/ml. Slides were then washed 3 times in tap water and mounted for microscopic examination.

### EXAMPLE 6

Testing of Heterologous Genes

Recombinant adenoviral vectors were prepared for injection into human skin grafts. Some of the vectors were engineered to express growth substances and cytokines; these are shown in Table 1. With two exceptions, KGF and FGF-10, all genes were of human origin. Other vectors were engineered to express proteolytic enzymes, their inhibitors, the antisense uPA receptor, oncogenes, tumor suppressor genes, adhesion receptors, and a pigment-related transcription substance. They are listed in Table 2.

Staining for β-galactosidase 3 days after injection of a lacZ control vector revealed that dermis cells were infected (stained), whereas the epidermal cells were not stained. Injection of the 100 μl virus suspended in PBS had created a defect that had largely closed within 2 to 3 days. A mild inflammatory infiltrate with monocytes/macrophages and neutrophils was invariably seen 3 days after injection. The injection of PBS also created a defect in the dermis but did not cause an inflammatory reaction. Histological changes in the human skin grafts after injection of viral vectors were evaluated using four response criteria: inflammation, neovascularization, connective tissue formation, and epidermal reaction.

### EXAMPLE 7

Inflammatory Response.

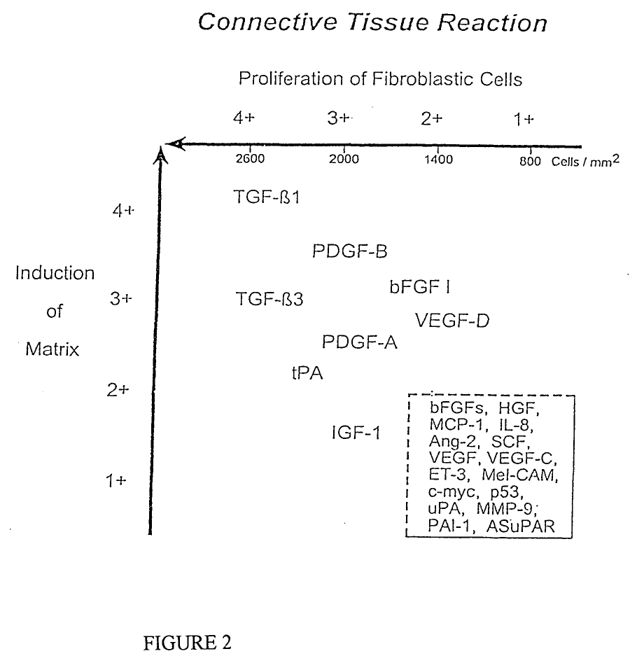

The inflammatory response was evaluated as thrombosis with concomitant necrosis and was quantitated as the number of thrombosed vessels per mm² (FIG. 2). A second criterion was the infiltration of inflammatory cells including neotrophillic granulocytes and monocytic cells. The cytokines with chemoattractive activity for monocytes/macrophages, MCP-1, and neutrophils, IL-8, were among the strongest inducers of an inflammatory response. Examples of neutrophil infiltration after IL-8 overexpression are given in FIG. 3A, and for monocyte infiltration after MCP-1 induction infiltration in FIG. 3B. A similarly strong reaction was induced by the serine protease uPA (but not tPA) and the inhibitor for PA, PAI-1. Thrombosis with a mild inflammatory cell infiltrate was observed with the antisense vector for the receptor for uPA, ASuPAR. The matrix in (matrix metalloprotease), MMP-9, induced strong thrombosis but little infiltration. The proto-oncogene c-myc and the growth substances, angiopoietin-2 and TGF-β1, were also positive when compared to control viral vectors. All other growth substances did not induce an inflammatory response, including bFGFs, PDGFs, VEGFs, IGF-1, Ang-1, ET-3, TGF-β3, any of the antisense vectors listed in Table 1. Adhesion receptors and oncogenes and tumor suppressor genes also did not induce significant reactivities.

### EXAMPLE 8

Vascular Response.

The strongest responses in host vessel formation and pericyte proliferation were seen for the serine proteases tPA and uPA. The response after tPA and uPA overexpressing in the grafts were more pronounced than for the growth substances VEGFs and angiopoietins. Angiogenic responses were also observed for the growth substances SCF, PDGF-A, TGF-β1, ET-3, and PDGF-B. Of all adhesion receptors over-expressed in human skin with an adenoviral vector, only Mel-CAM induced both vessel formation and pericyte proliferation. There was limited response after bFGF, IGF-1, MCP-1, IL-8, or TGF-β3 overexpression. However, after repeat (3 injections) of bFGF within a strong angiogenesis and stroma formation were produced. It appears that gene products that are slowly released from cells for activation of the cellular environment may require repeated injections. C-myc, MMP-9, ASuPAR, or PAI-1 did not induce any significant changes after a single injection of the adenoviral vector.

### EXAMPLE 9

Connective Tissue Reaction

Stimulation of the dermal stroma was characterized by the induction of proliferation of fibroblastic cells and the extent of matrix formation. Matrix formation was quantified by confocal microscopy after staining with trichrome. The strongest inducer of both fibroblast growth and matrix formation was the vector for the growth substance TGF-β1, followed by PDGF-B, TGF-β3, bFGFL, VEGF-D, PDGF A and IGF-1.

### EXAMPLE 10

Epidermal Reaction.

Several of the injected adenoviral vectors induced a strong response of the epidermis. This could only be achieved through indirect mechanisms of dermal cells producing stimulatory substances because the adenoviruses infected only the dermal and not epidermal cells (Liechty, 1999, Sylvester, 2000). The strongest response was induced by ASuPAR, followed by SCF and ET-3. Of other growth substances and cytokines PDGF-B, VEGF-D, bFGFL, MCP-1 and IL-8 induced a keratinocyte growth response and remodeling of the epidermis by increased papillomatosis. A response was also seen with the c-myc and p53 vectors.

### EXAMPLE 11

Cell Lines

Human dermal fibroblasts (FF2441) were isolated as explant cultures from typsin-treated and epidermis-stripped neonatal foreskins and maintained in DMEM supplemented with 10% FBS. Human microvascular endothelial cells (HMVEC) originated from normal dermis of adult human skin surgical specimen and were kindly provided by Dr. Doug Fraker, University of Pennsylvania, Philadelphia, Pa., USA. These cells were isolated and characterized as previously described. All cells and three dimensional reconstructs were incubated at 37° C. in 98% humidified air containing 5% CO₂.

### EXAMPLE 12

Antibodies, Fluorescent Stains and Chemical Reagents

Anti-human antibodies against P3, PECAM, b3, Id1, Id3, P-Selectin, Ki-67, KDR, von Willebrand Factor (νWF) (mouse anti-human monoclonal IgG1), are known, commercially available antibodies. Iodide fluorescent nuclear stains, L-glutamine, Heparin, sodium bicarbonate and other commonly utilized research chemical reagents were obtained from Sigma Chemical Co. (St. Louis, Mo.). DiI was obtained from Molecular Probes.

### EXAMPLE 13

Inverted Fluorescent Microscopy (40×) of HMVEC Migrating into Collagen I Gel Supported by Fibroblasts in Complete Medium at 48 hr. Incubation

An HMVEC monolayer cultured to 80% confluency in a collagen-coated 24 well plate was stained with DiI (red) and incubated for 24 hr. Subsequently a dermal reconstruct was constructed overlying the monolayer. After 24 hr. of incubation with complete medium supplementation, the collagen gel was fixed with Prefer, counterstained with Hoechst (blue), removed from the plate, and the whole gel was mounted on a slide. The blue nuclei of fibroblasts (not stained with DiI) can be observed surrounding the endothelial cell with its red-stained cell membrane that has migrated into the gel and curled into an elongated, tubular morphology with branching cytoplasmic extensions.

### EXAMPLE 14

Inverted Fluorescent Microscopy (100×) of HMVEC Proliferating Within Collagen I Gel Supported by Fibroblasts in Complete Medium at 5 Days of Incubation

An HMVEC monolayer cultured to 80% confluency in a collagen-coated 24 well plate. Subsequently a dermal reconstruct was constructed overlying the monolayer. After 5 days of incubation with complete medium supplementation every 24 hr, the collagen gel was fixed with Prefer, counterstained with Hoechst (blue), and double stained with anti-human vWF VIII (green) and Ki67 (red), removed from the plate, and the whole gel was mounted on a slide. The nucleus (blue) of an endothelial cell (green cell membrane) were positive for the proliferation marker Ki67 (red).

### EXAMPLE 15

Anti-Human PECAM Immunohistochemistry Examination of a Cross Section of a Gel Showing Endothelial Cells Differentiated into “Capillary-like” Networks, 4×

An HMVEC monolayer cultured to 80% confluency in a collagen-coated 24 well plate. Subsequently a dermal reconstruct was constructed overlying the monolayer. After 5 days, and also after 11 days, of incubation with complete media supplementation every 48 hr, the collagen gel was removed from the plate, fixed with Prefer, and embed in paraffin. 5 μm sections of the gel were placed on a slide and stained by anti-human PECAM immunohistochemistry. The endothelial cells (brown stain) were observed to have migrated into the gel and differentiated into a network of interconnecting hollow tubes.

### EXAMPLE 16

Fluorescence Confocal Microscopy of a Whole Mounted Gel Outlining the Lumen of Branching Tubes with Anti-vWF VIII Immunofluorescence, 10×

An HMVEC monolayer cultured to 80% confluency in a collagen-coated 24 well plate. Subsequently a dermal reconstruct was constructed overlying the monolayer. After 5 days of incubation with complete media supplementation every 48 hr, the collagen gel was removed from the plate, fixed with Prefer, counterstained with Hoechst, and the gel was incubated under gentle swirling/shaking conditions with anti-human vWF VIII (green) The endothelial cells (green) were observed to have migrated into the gel and many have differentiated into a network of interconnecting hollow tubes. Some were seen as scattered cells within the gel.

### EXAMPLE 17

Fluorescence Confocal Microscopy with Three Dimensional Reconstructions of a Whole Mounted Gel Outlining the VEGF-Induced Branching Cord-like Morphology of Endothelial Cells With Anti-vWF VIII Immunofluorescence, 20×

An HMVEC monolayer cultured to 80% confluency in a collagen-coated 24 well plate. Subsequently a dermal reconstruct was constructed overlying the monolayer. 24 hrs prior to the construction of the dermal reconstruct, the fibroblasts used to mix within the collagen gel were transduced with (A) LacZ and (B) VEGF adenovirus vector @ 20 pfu. The collagen gel and the incubation medium contained reduced serum (1%). After 5 days of incubation with reduced media supplementation every 48 hr, the collagen gel was removed from the plate, fixed with Prefer, counterstained with Hoechst, and the gel was incubated under gentle swirling/shaking conditions with anti-human vWF VIII (green). The gel was removed from be plate and mounted whole on a slide. The endothelial cells (green) migrated into the gel and differentiated into interconnecting cords. No definitive hollow tubes or luminal space were identified. Findings were confirmed by PECAM immunohistochemistry.

### EXAMPLE 18

Construction and Maintenance of Three-Dimensional “Vascularized” Artificial Dermal Reconstructs

Acellular and cellular collagen layers were separately created by mixing the following components, in the same order and volume-ratios as listed below.

Acellular collagen layer 0.59 mL 10×M199 (Gibco BRL/Life Technologies Inc., Baltimore, Md., supplemented with L-Glutamine (200M), Heparin (100 U/mL, all from) and vitamin C (50 mg/mL)), 0.05 mL L-glutamine (200 mM), 0.6 mL (10%) Fetal Bovine Serum, 0.17 mL Sodium Bicarbonate, 4.6 mL Bovine Collagen type I (Organogenesis, Canton, Mass., Concentration 1 mg/mL.

Cellular collagen layer: 0.59 mL 10×M199 (Gibco BRL/Life Technologies Inc., Baltimore, Md., supplemented with L-Glutamine (200M), Heparin (100 U/mL) and vitamin C (50 mg/mL)), 0.05 mL L-glutamine, 0.6 mL (10%) Fetal Bovine Serum, 0.17 mL Sodium Bicarbonate, 4.6 mL Bovine Collagen type I, and 500,000 fibroblasts/mL.

Complete Medium: modified MCDB 131 supplemented with Bovine brain extract (0.012 mg/mL, 0.4%), recombinant human Endothelial Growth Factor (0.01 g/mL, 0.1%), fetal bovine serum (5.0%), gentamycin (0.001 mg/ml, 0.1%) and hydrocortisone (0.001 mg/mL, 0.1%). All these media components were purchased from BioWhittaker, Walkzrsville, Md. (“EGM-MV Bullet” catalogue # cc3121 (media) & cc 4143 (components)).

When adenoviral vectors were utilized the composition of the collagen mixture was adjusted such as to reduced fetal bovine serum to 1% and “complete media”, detailed above, was changed to “reduced media”, consisting of modified MCDB 131 supplemented with Bovine brain extract (0.0012 mg/mL, 0.04%), recombinant human Endothelial Growth Factor (0.001 g/mL, 0.01%), fetal bovine serum (1.0%), gentamycin (0.0001 mg/ml, 0.1%) and hydrocortisone (0.001 mg/mL, 0.1%)

Constructions of ‘dermis-like’ or ‘dermal’ reconstruct overlying an endothelial cell monolayer: All the components of the above layers were kept on ice. The acellular matrix was created first by mixing all the components and then 150 μL of the liquid mixture was added to the surface of an endothelial cell monolayer, previously cultured to 80% confluency on a 24 well collagen coated plate. The maintenance medium for the endothelial monolayer was removed prior to adding the acellular collagen matrix. The plate was allowed to remain at room temperature, for 10 minutes, to allow for polymerizing of the acellular layer. While the acellular layer was polymerizing, the fibroblasts to be used within the cellular layer were trypsinized and resuspended within the cellular collagen mixture to create a final cell density of 500,000 cells/mL. When adenoviral vectors were used, the fibroblasts were transfected 24 hrs prior to creating the reconstruct. 450 μL of the cellular mix was added over the polymerized acellular mix. The plate was incubated for 10 minutes at 37° C. in 98% humidified air containing 5% CO₂, to allow for polymerization to occur. Then 2.5 ml of medium was added to the polymerized reconstruct, and the plate was again incubated for the length of the experiment. Medium was replenished (2.5 ml) every 48 hrs.

### EXAMPLE 19

Production and Maintenance of Dermal Reconstructs Containing Melanoma Cells

Skin reconstructs were prepared essentially as described with modifications (Meier et al., Am. J. Pathol. 156:193 (2000)). Human fibroblasts (FF2441) were added to neutralized bovine type I collagen (Organogenesis, Canton, Mass.) to a final concentration of 0.8-1 mg/ml of collagen in MEM (Bio Whittaker, Walkersville, Mass.), 1.66 mM L-glutamine (Life Technologies), 10% FBS and 0.21% sodium bicarbonate (Biowhittaker). Three ml of fibroblast-containing collagen (2.5×10⁴ cells/ml) were added to each insert of a 6-well tissue-culture tray (Organogenesis) after precoating with 1 ml of acellular collagen. Mixtures were allowed to constrict in DMEM with 10% FBS for 5-7 days. Human primary and metastatic melanoma cells were isolated from clinically and histologically defined lesions and cultured as described (Herlyn et al., Cancer Res. 45:5670 (1985); Hsu et al., The Wistar melanoma (WM) cell lines. In: J. R. W. Masters and B. Palsson (eds.), Human Cell Culture, vol 3, Solid Cancers, pp 259-274. Norwell, Mass., Kluwer Academic Publishers, (1999). They were maintained in MCDB153 with 20% Leibovitz's L-15 medium (Life Technologies), 2% FBS, and 5 mg/ml insulin (Sigma). Keratinocytes were mixed with melanoma cells at a ratio of 5:1 to 10:1 in low-calcium epidermal growth medium containing DMEM, F-12 Ham's (Life Technologies), 1% newborn calf serum (Hyclone), 4 mM glutamine, 1.48×10⁻⁶ M hydrocortisone, 4 pM progesterone, 20 pM triiodothyronine, 0.1 mM O-phosphoryletlianolamine, 0.18 mM adenine (Sigma), 5 mg/ml insulin, 5 mg/ml transferrin, 5 mM ethanolamine, 5 g/ml selenium (Biowhittaker, Walkersville, Md.) and 50 μg/ml gentamycin (Mediatech, Hemdon, Va.). A total of 5-6×10⁵ cells was seeded on each contracted collagen gel. Cultures were maintained submerged in low calcium growth medium for 2 days and in normal calcium (1.88 mM) growth medium for another 2 days and then raised to the air-liquid interface for 10-12 days with feeding from below with normal calcium and high-serum (20%) medium.

References to Nucleic Acid Sequences of the Invention:

Tissue Plasminogen Activator

1. Harris,T. J., Patel,T., Marston,F. A., Little,S., Emtage,J. S., Opdenakker,G., Volckaert,G., Rombauts,W., Billiau,A. and De Somer,P. Cloning of cDNA coding for human tissue-type plasminogen activator and its expression in Escherichia coli. Mol. Biol. Med. 3 (3), 279-292 (1986). NM_{—}000930

2. Niwa,M., Saito,Y., Sasaki,H., Hayashi,M. and Otsuka,K. cDNA Of Human Tissual Plasminogen Activating Factor. Patent: JP 1989174388-A 4Jul. 10,1989-. E02027

3. Niwa,M., Saito,Y., Sasaki,H., Hayashi,M. and Otsuka,K. cDNA Of Human Tissual Plasminogen Activating Factor. Patent: JP 1989174388-A 2JUL.10,1989. E02025

4. Harris,T. J., Patel,T., Marston,F. A., Little,S., Emtage,J. S., Opdenakker,G., Volckaert,G., Rombauts,W., Billiau,A. and De Somer,P. Cloning of cDNA coding for human tissue-type plasminogen activator and its expression in Escherichia coli. Mol. Biol. Med. 3 (3), 279-292 (1986). M15518.

Plasminogen Activator Inhibitor

1. Wun,T. C. and Kretzmer,K. K. cDNA cloning and expression in E. coli of a plasminogen activator inhibitor (PAI) related to a PAI produced by Hep G2 hepatoma cell. FEBS Lett. 210 (1), 11-16 (1987). X04744.

2. Ginsburg,D., Zeheb,R., Yang,A. Y., Rafferty,U. M., Andreasen,P. A., Nielsen,L., Dano,K., Lebo,R. V. and Gelehrter,T. D. cDNA cloning of human plasminogen activator-inhibitor from endothelial cells. J. Clin. Invest. 78 (6), 1673-1680 (1986). M16006.

Fibroblast Growth Factor Binding Protein (HBP17)

1. Wu,D. Q., Kan,M. K., Sato,G. H., Okamoto,T. and Sato,J. D. Characterization and molecular cloning of a putative binding protein for heparin-binding growth factors. J. Biol. Chem. 266 (25), 16778-16785 (1991). NM_{—}005130

2. Wu,D. Q., Kan,M. K., Sato,G. H., Okamoto,T. and Sato,J. D. Charactcrization and molecular cloning of a putative binding protein for heparin-binding growth factors. J. Biol. Chem. 266 (25), 16778-16785 (1991). M60047

Micropthalmia Associated Transcription Factor (MITF)

1. Tachibana,M., Perez-Jurado,L. A., Nakayama,A., Hodgkinson,C. A., Li,X., Schneider,M., Miki,T., Fex,J., Francke,U. and Amheiter,H. Cloning of MITF, the human homolog of the mouse microphthalmia gene and assignment to chromosome 3p14.1-p12.3 Hum. Mol. Genet. 3 (4), 553-557 (1994). NM_{—}000248.

2. Amae,S., Fuse,N., Yasumoto,K., Sato,S., Yajima,I., Yamamoto,H., Udono,T., Durlu,Y. K, Tamai,M., Takahashi,K. and Shibahara,S. Identification of a novel isoform of microphthalmaia-associated transcription factor that is enriched in retinal pigment epithelium Biochem. Biophys. Res. Commun. 247 (3), 710-715 (1998). AB006909.

β-Galactosidase (LacZ)

1. Blattner,F. R., Plunkett,G. III, Bloch,C. A., Perna,N. T., Burland,V., Riley,M., Collado-Vides, J., Glasner,J. D., Rode,C. K., Mayhew,G. F., Gregor,J., Davis,N. W., Kirkpatrick,H. A., Goeden,M. A., Rose,D. J., Mau,B. and Shao,Y. The complete genome sequence of Escherichia coli K-12. Science 277(5331), 1453-1474 (1997). AE000141.

Green Fluorescence Protein (GFP)

1. Inouye,S. and Tsuji,F. I. Aequorea green fluorescent protein. Expression of the gene and fluorescence characteristics of the recombinant protein. FEBS Lett. 341 (2-3), 277-280 (1994). L29345.

Matrix Metalloproteinase-9 (MMP-9)

1. Wilhelm,S. M., Collier,I. E., Marmer,B. L., Eisen,A. Z., Grant,G. A. and Goldberg,G. I. SV40-transformed human lung fibroblasts secrete a 92-kDa type IV collagenase which is identical to that secreted by normal human macrophages [published erratum appears in J Biol Chem Dec. 25, 1990; 265(36):22570]. J. Biol. Chem. 264 (29), 17213-17221 (1989). NM_{—}004994

Tissue Inhibitor of Matrix Metalloproteinase-1 (TIMP-1)

1. Gasson,J. C., Golde,D. W., Kaufman,S. E., Westbrook,C. A., Hewick,R. M., Kaufman,R. J., Wong,G. G., Temple,P. A., Leary,A. C., Brown,E. L., Orr,E. C. and Clark,S. C. Molecular characterization and expression of the gene encoding human erythroid-potentiating activity. Nature 315 (6022), 768-771 (1985). NM_{—}003254

2. Docherty,A. J., Lyons,A., Smith,B. J., Wright,E. M., Stephens,P. E., Harris,T. J., Murphy,G. and Reynolds,J. J. Sequence of human tissue inhibitor of metalloproteinases and its identity to erythroid-potentiating activity. Nature 318 (6041), 66-69 (1985). NM_{—}003254

3. Carmichael,D. F., Sommer,A., Thompson,R. C., Anderson,D. C., Smith,C. G., Welgus,H. G. and Stricklin,G. P. Primary structure and cDNA cloning of human fibroblast collagenase inhibitor. Proc. Natl. Acad. Sci. U.S.A. 83 (8), 2407-2411 (1986). NM_{—}003254

4. Huebner,K., Isobe,M., Gasson,J. C., Golde,D. W. and Croce,C. M. Localization of the gene encoding human erythroid-potentiating activity to chromosome region Xp11.1-Xp11.4 Am. J. Hum. Genet. 38 (6), 819-826 (1986). NM_{—}003254

5. Willard,H. F., Durfy,S. J., Mahtani,M. M., Dorkins,H., Davies,K. E. and Williams,B. R. Regional localization of the TIMP gene on the human X chromosome. Extension of a conserved synteny and linkage group on proximal Xp. Hum. Genet. 81 (3), 234-238 (1989). NM_{—}003254

6. Rapp G, Freudenstein J, Klaudiny J, Mucha J, Wempe F, Zimmer M and Scheit K H. Characterization of three abundant mRNAs from human ovarian granulosa cells. DNA Cell Biol. 9 (7), 479-485 (1990). NM_{—}003254

7. Osthues,A., Knauper,V., Oberhoff,R., Reinke,H. and Tscheschle,H Isolation and characterization of tissue inhibitors of metalloproteinases (TIMP-1 and TIMP-2) from human rheumatoid synovial fluid. FEBS Lett. 296 (1), 16-20 (1992). NM_{—}003254

8. Opbroek,A., Kenney,M. C. and Brown,D. Characterization of a human corneal metalloproteinase inhibitor (TIMP-1). Curr. Eye Res. 12 (10), 877-883 (1993). NM_{—}003254

Urokinase Plasminogen Activator (uPA)

1. Thrombus-Specific Antibody Derivatives. Patent: WO 9116353-A 36 -OCT -31, 1991. A18397.

2. Cassani,G., Robbiati,F. M., Blasi,F., Nolli,M. and Corti,A. Method for preparing single chain urokinase. JOURNAL Patent: EP 0303028-A 1 15-FEB. 15,1989; Gruppo Lepetit S.P.A.; Gruppo Lepetit S.p.A. A11211.

3. Roldan,A. L., Cubellis,M. V., Masucci,M. T., Behrendt,N., Lund,L. R., Dano,K., Appella,E. and Blasi,F. Cloning and expression of the receptor for human urokinase plasminogen activator, a central molecule in cell surface, plasmin dependent proteolysis. EMBO J. 9, 467-474 (1990). U08839.

Beta3-Integrin

1. Zimrin,A. B., Eisman,R., Vilaire,G., Schwartz,E., Bennett,J. S. and Poncz,M. Structure of platelet glycoprotein IIIa. A common subunit for two different membrane receptors. J. Clin. Invest. 81 (5), 1470-1475 (1988). NM_{—}000212.

2. Fitzgerald,L. A., Steiner,B., Rall,S. C. Jr., Lo,S. S. and Phillips,D. R. Protein sequence of endothelial glycoprotein IIIa derived from a cDNA clone. Identity with platelet glycoprotein IIIa and similarity to ‘integrin’. J. Biol. Chem. 262 (9), 3936-3939 (1987). J02703.

3. Frachet,P., Uzan,G., Thevenon,D., Denarier,E., Prandini,M. H. and Marguerie,G. GPIIb and GPIIa amino acid sequences deduced from human megakaryocyte cDNAs. Mol. Biol. Rep. 14, 27-33 (1990). M35999.

MUC18 (Mel-CAM)

1. Lehmann J M, Riethmuller G and Johnson J P. MUC18, a marker of tumor progression in human melanoma, shows sequence similarity to the neural cell adhesion molecules of the immunoglobulin superfamily. Proc. Natl. Acad. Sci. U.S.A. 86 (24), 9891-9895 (1989). NM_{—}006500.

2. Gui,L., Chang,L., Browne,P. V. and Hebbel,R. P. P1H12 from human umbilical vein endothelial cells. JOURNAL Unpublished. AF089868.

E-Cadherin

1. Shibamoto,S. and Fukudome,Y. Direct Submission JOURNAL Submitted (MAR. 19,1999) to the DDBJ/EMBL/GenBank databases. Sayumi Shibamoto, Setsunan University, Faculty of Pharmaceutical Sciences,Department of Biochemistry; 45-1 Nagaotoge-cho, Hirakata, Osaka 573-0101, Japan. AB025106.

2. Bussemakers,M. J., van Bokhoven,A., Mees,S. G., Kemler,R. and Schalken,J. A. Molecular cloning and characterization of the human E-cadherin cDNA. Mol. Biol. Rep. 17 (2), 123-128 (1993). Z13009.

3. Rimm,D. L. and Morrow,J. S. Molecular cloning of human E-cadherin suggests a novel subdivision of the cadherin superfamily. Biochem. Biophys. Res. Commun. 200 (3), 1754-1761 (1994). L08599.

N-Cadherin

1. Walsh,F. S., Barton,C. H., Putt,W., Moore,S. E., Kelsell,D., Spurr,N. and Goodfellow,P. N. N-cadherin gene maps to human chromosome 18 and is not linked to the E-cadherin gene. J. Neurochem. 55 (3), 805-812 (1990). M34064.

Alpha2 Integrin

1. Takada,Y. and Hemler,M. E. The primary structure of the VLA-2/collagen receptor alpha 2 subunit (platelet GPIa): homology to other integrins and the presence of a possible collagen-binding domain. J. Cell Biol. 109 (1), 397-407 (1989). NM_{—}002203.

Alpha5 Interin

1. Argraves,W. S., Suzuki,S., Arai,H., Thompson,K., Pierschbacher,M. D. and Ruoslahti,E. Amino acid sequence of the human fibronectin receptor. J. Cell Biol. 105 (3), 1183-1190 (1987). NM_{—}002205.

2. Coulombel,L., Auffray,I., Gaugler,M. H. and Rosemblatt,M. Expression and function of integrins on hematopoictic progenitor cells. Acta Haematol. 97 (1-2), 13-21 (1997). NM_{—}002205.

Human Papillomavirus Type 16 E6 and E7 Oncogene

1. Seedorf,K., Kraemmer,G., Duerst,M., Suhai,S. and Roewekamp,W. G. Human papillomavirus type 16 DNA sequence. Virology 145, 181-185 (1985). NC_{—}001526.

2. Tornesello,M. L., Buonaguro,F. M., Meglio,A., Buonaguro,L., Beth-Giraldo,E. and Giraldo,G. Sequence variations and viral genomic state of human papillomavirus type 16 in penile carcinomas from Ugandan patients. J. Gen. Virol. 78 (Pt 9), 2199-2208 (1997). AF003019.

3. Seedorf.,K, Kraemmer,G., Duerst,M., Suhai,S. and Roewekamp,W. G. Human papillomavirus type 16 DNA sequence. Virology 145, 181-185 (1985). K02718.

c-Myc

1. Gazin,C., Dupont de Dinechin,S., Hampe,A., Masson,J. M., Martin,P., Stehelin,D. and Galibert,F. Nucleotide sequence of the human c-myc locus: provocative open reading frame within the first exon. EMBO J. 3 (2), 383-387 (1984). X00364 J00120 K01908 V00501.

2. Colby,W. W., Chen,E. Y., Smith,D. H. and Levinson,A. D. Identification and nucleotide sequence of a human locus homologous to the v-myc oncogene of avian myelocytomatosis virus MC29. Nature 301 (5902), 722-725 (1983). X00364 J00120 K01908 V00501.

3. Saito,H., Hayday,A. C., Wiman,K., Hayward,W. S. and Tonegawa,S. Activation of the c-myc gene by translocation: a model for translational control Proc. Natl. Acad. Sci. U.S.A. 80 (24), 7476-7480 (1983). X00364 J00120 K01908 V00501.

4. Gazin,C., Rigolet,M., Briand,J. P., Van Regenmortel,M. H. and Galibert,F. Immunochemical detection of proteins related to the human c-myc exon 1. EMBO J. 5 (9), 2241-2250 (1986). X00364 J00120 K01908 V00501.

P16 Tumor Suppressor Protein

1. Serrano,M., Hannon,G. J. and Beach,D. A new regulatory motif in cell-cycle control causing specific inhibition of cyclin D/CDK4 Nature 366 (6456), 704-707 (1993). NM_{—}000077.

2. Duro,D., Bernard,O., Della Valle,V., Berger,R. and Larsen,C. J. A new type of p16INK4/MTS1 gene transcript expressed in B-cell malignancies. Oncogene 11 (1), 21-29 (1995). U26727.

P21 Tumor Suppressor Protein

1. Use of a sensitive and efficient subtraction hybridization protocol for the identification of genes differentially regulated during the induction of differentiation in human melanoma cells. Jiang,H. and Fisher,P. B. Mol. Cell. Differ. 1, 285-299 (1993). U09579.

2. Harper,J. W., Adarmi,G. R., Wei,N., Keyomarsi,K. and Elledge,S. J. The p21 Cdk-interacting protein Cip1 is a potent inhibitor of G1 cyclin-dependent kinases. Cell 75 (4), 805-816 (1993). L25610.

P53 Tumor Suppressor

1. Matlashewski,G., Lamb,P., Pim,D., Peacock,J., Crawford,L. and Benchimol,S. Isolation and characterization of a human p53 cDNA clone: expression of the human p53 gene. EMBO J. 3 (13), 3257-3262 (1984). NM_{—}000546.

2. Zakut-Houri,R., Bienz-Tadmor,B., Givol,D. and Oren,M. Human p53 cellular tumor antigen: cDNA sequence and expression in COS cells. EMBO J. 4 (5), 1251-1255 (1985). NM_{—}000546.

3. Harlow,E., Williamson,N. M., Ralston,R., Helfman,D. M. and Adams,T. E. Molecular cloning and in vitro expression of a cDNA clone for human cellular tumor antigen p53. Mol. Cell. Biol. 5 (7), 1601-1610 (1985). NM_{—}000546.

4. Lamb,P. and Crawford,L. Characterization of the human p53 gene. Mol. Cell. Biol. 6 (5), 1379-1385 (1986). NM_{—}000546.

5. Buchman,V. L., Chumakov,P. M., Ninkina,N. N., Samarina,O. P. and Georgiev,G. P. A variation in the structure of the protein-coding region of the human p53 gene. Gene 70 (2), 245-252 (1988). NM_{—}000546.

Antisense Cyclin D1

1. Xiong,Y., Connolly,T., Futcher,B. and Beach,D. Human D-type cyclin Cell 65 (4), 691-699 (1991). NM_{—}001758.

Basic Fibroblast Growth Factor (18 kDA)

1. Itoh,N., Terachi,T., Ohta,M. and Seo,M. K. The complete amino acid sequence of the shorter form of human basic fibroblast growth factor receptor deduced from its cDNA. Biochem. Biophys. Res. Commun. 169 (2), 680-685 (1990). M37722.

Basic Fibroblast Growth Factor (18, 22, 23, 24 kDA)

1. Prats,H., Kaghad,M., Prats,A. C., Klagsbrun,M., Lelias,J. M., Liauzun,P., Chalon,P., Tauber,J. P., Amalric,F., Smith,J. A. and Caput,D. High molecular mass forms of basic fibroblast growth factor are initiated by alternative CUG codons Proc. Natl. Acad. Sci. U.S.A. 86 (6), 1836-1840 (1989). J04513.

Platelet Derived Growth Factor A

1. Betsholtz C, Johnsson A, Heldin C H, Westermark B, Lind P, Urdea M S, Eddy R, Shows T B, Philpott K, Mellor A L and et al. cDNA sequence and chromosomal localization of human platelet-derived growth factor A-chain and its expression in tumour cell lines. Nature 320 (6064), 695-699 (1986). NM_{—}002607.

Platelet Derived Growth Factor B

1. Collins,T., Ginsburg,D., Boss,J. M., Orkin,S. H. and Pober,J. S. Cultured human endothelial cells express platelet-derived growth factor B chain: cDNA cloning and structural analysis. Nature 316 (6030), 748-750 (1985). X02811.

Insulin-like Growth Factor-I

1. Jansen,M., van Schaik,F. M., Ricker,A. T., Bullock,B., Woods,D. E., Gabbay,K. H., Nussbaum,A. L., Sussenbach,J. S. and Van den Brande,J. L. Sequence of cDNA encoding human insulin-like growth factor I precursor. Nature 306 (5943), 609-611 (1983). X00173.

Monocyte Chemoattractant Protein-1

1. Li,Y. S., Shyy,Y. J., Wright,J. G., Valente,A. J., Cornhill,J. F. and Kolattukudy,P. E. The expression of monocyte chemotactic protein (MCP-1) in human vascular endothelium in vitro and in vivo. Mol. Cell. Biochem. 126 (1), 61-68 (1993). S69738.

2. Yoshimura,T. and Leonard,E. J. Human monocyte chemoattractant protein-1 (MCP-1) Adv. Exp. Med. Biol. 305,47-56 (1991). S71513.

Interleukin-8

1. Schmid,J. and Weissmann,C. Induction of mRNA for a serine protease and a beta-thromboglobulin-like protein in mitogen-stimulated human leukocytes J. Immunol. 139, 250-256 (1987). M17017.

2. Walz,A., Burgener,R., Car,B., Baggiolini,M., Kunkel,S. L. and Strieter,R. M. Structure and neutrophil-activating properties of a novel inflammatory peptide (ENA-78) with homology to Interleukin 8 J. Exp. Med. 174 (6), 1355-1362 (1991). L37036 Z46254.

Vascular Endothelial Growth Factor D

1. Yamada Y, Nezu J, Shimane M and Hirata Y. Molecular cloning of a novel vascular endothelial growth factor, VEGF-D Genomics 42 (3), 48348 (1997). NM_{—}004469.

2. Achen,M. G., Jeltsch,M., Kukk,E., Makinen,T., Vitali,A., Wilks,A. F., Alitalo,K. and Stacker,S. A. Vascular endothelial growth factor D (VEGF-D) is a ligand for the tyrosine kinases VEGF receptor 2 (Flk1) and VEGF receptor 3 (Flt4) Proc. Natl. Acad. Sci. U.S.A. 95 (2), 548-553 (1998). AJ00185.

3. Yamada,Y., Nezu,J., Shiimane,M. and Hirata,Y. Molecular cloning of a novel vascular endothelial growth factor, VEGF-D Genomics 42 (3), 483488 (1997). D89630.

Vascular Endothelial Growth Factor

1. AIDS-associated Kaposi's sarcoma cells in culture express vascular endothelial growth factor Biochem. Biophys. Res. Commun. 183 (3), 1167-1174 (1992). NM_{—}003376.

2. Leung,D. W., Cachianes,G., Kuang,W.-J., Goeddel,D. V. and Ferrara,N. Vascular endothelial growth factor is a secreted angiogenic mitogen Science 246, 1306-1309 (1989). M32977.

Vascular Endothelial Growth Factor C

1. Joukov,V., Pajusola,K., Kaipainen,A., Chilov,D., Lahtinen,I., Kukk,E., Saksela,O., Kalkkinen,N. and Alitalo,K. A novel vascular endothelial growth factor, VEGF-C, is a ligand for the Flt4 (VEGFR-3) and KDR (VEGFR-2) receptor tyrosine kinases EMBO J. 15 (2), 290-298 (1996). X94216.

Pleiotrophin

1. Masuda,H., Tsujimura,A., Yoshioka,M., Arai,Y., Kuboki,Y., Mukai,T., Nakamura,T., Tsuji,H., Nakagawa,M. and Hashimoto-Gotoh,T. Bone mass loss due to estrogen deficiency is compensated in transgenic mice overexpressing human osteoblast stimulating factor-1 Biochem. Biophys. Res. Commun. 238 (2), 528-533 (1997). NM_{—}002825.

Transforming Growth Factor-Betal

1. Derynck,R., Jarrett, J. A., Chen,E. Y., Eaton,D. H., Bell,J. R., Assoian,R. K., Roberts,A. B., Sporn,M. B. and Goeddel,D. V. Human transforming growth factor-beta complementary DNA sequence and expression in normal and transformed cells Nature 316 (6030), 701-705 (1985). X02812 J05114.

Transforming Growth Factor-Beta3

1. Derynck, R., Lindquist,P. B., Lee,A., Wen,D., Tamm,J., Graycar,J. L., Rhee,L., Mason,A. J., Miller,D. A., Coffey,R. J., Moses,H. L. and Chen,E. Y. A new type of transforming growth factor-beta, TGF-beta 3. EMBO J. 7 (12), 3737-3743 (1988). NM_{—}003239.

Hepatocyte Growth Factor

1. Weidner,K. M., Arakaki,N., Hartmann,G., Vandekerckhove,J. S., Weingart,S., Rieder,H., Fonatsch,C., Tsubouchi,H., Hishida,T., Daikuhara,Y. and Birchmeier,W. Evidence for the identity of human scatter factor and human hepatocyte growth factor Proc. Natl. Acad. Sci. U.S.A. 88 (16), 7001-7005 (1991). M73240.

Angiopoietitn 1

1. Nomura,N., Miyajima,N., Sazuka,T., Tanaka,A., Kawarabayasi,Y., Sato,S., Nagase,T., Seki,N., Ishikawa,K. and Tabata,S. Prediction of the coding sequences of unidentified human genes. I. The coding sequences of 40 new genes (KIAA0001-KIAA0040) deduced by analysis of randomly sampled cDNA clones from human immature myeloid cell line KG-1 (supplement) DNA Res. 1 (1), 47-56 (1994). NM_{—}001146.

2. Davis,S., Aldrich,T. H., Jones,P. F., Acheson,A., Compton,D. L., Jain,V., Ryan,T. E., Bruno,J., Radziejewski,C., Maisonpierre,P. C. and Yancopoulos,G. D. Isolation of angiopoietin-1, a ligand for the TIE2 receptor, by secretion-trap expression cloning Cell 87 (7), 1161-1169 (1996). U83508.

Angiopoietin 2

1. Maisonpierre,P. C., Suri,C., Jones,P. F., Bartunkova,S., Wiegand,S. J., Radziejewski,C., Compton,D., Aldrich,T. H., Papadopoulos,N., Daly,T. J., Davis,S., Sato,T. N. and Yancopoulos,G. D. Angiopoietin-2, a natural antagonist for tie2 that disrupts in vivo angiogenesis Science 277 (5322), 55-60 (1997). NM_{—}001147.

Stem Cell Factor

1. Martin,F. H., Suggs,S., Langley,K. E., Lu,H. S., Ting,J., Okino,K. H., Morris,C. F., McNiece,I. K., Jacobsen,F. W., Mendiaz,E. A., Birkett,N. C., Smith,K. A., Johnson,M. J., Parker,V. P., Flores,J. C., Patel,A. C., Fisher,E. F., Erjavec,H. O., Herrera,C., Wypych,J., Sachdev,R. K., Pope,J. A., Leslie,I., Wen,D., Lin,C. -H., Cupples,R. L. and Zsebo,K. M. Primary structure and functional expression of rat and human stem cell factor DNAs Cell 63 (1), 203-211 (1990). NM_{—}003994.

Endothelin-3

1. Inoue,A., Yanagisawa,M., Kimura,S., Kasuya,Y., Miyauchi,T., Goto,K. and Masaki, T. The human endothelin family: three structurally and pharmacologically distinct isopeptides predicted by three separate genes Proc. Natl. Acad. Sci. U.S.A. 86 (8), 2863-2867 (1989). NM_{—}000114.

Keratinocyte Growth Factor (FGF7)

1. Jones,M. L., Dato,M. E. and Greenberg,J. M. Regulation of KGF/FGF-7 Expression in Immortalized Clonal Mouse Fetal Lung Mesenchymal Cells JOURNAL Unpublished. U58503.

2. Mason,I. J., Fuller-Pace,F., Smith,R. and Dickson,C. FGF-7 (keratinocyte growth factor) expression during mouse development suggests roles in myogenesis, forebrain regionalisation and epithelial-mesenchymal interactions Mech. Dev. 45 (1), 15-30 (1994). NM_{—008008.}

Fibroblast Growth Factor 10

1. Tagashira,S., Harada,H., Katsumata,T., Itoh,N. and Nakatsuka,M. Cloning of mouse FGF10 and up-regulation of its gene expression during wound healing Gene 197 (1-2), 399-404 (1997). D89080.

References

1. Atillasoy E S, Seykora J T, Soballe P W, et al. UVB induces atypical melanocytic lesions and melanoma in human skin. Am J Pathol 1998; 152(5):1179-86.

2. Baker A H, Wilkinson G W, Hembry R M, Murphy G, Newby A C. Development of recombinant adenoviruses that drive high level expression of the human metalloproteinase-9 and tissue inhibitor of metalloproteinase-1 and -2 genes: characterization of their infection into rabbit smooth muscle cells and human MCF-7 adenocarcinoma cells. Matrix Biol 1996;15(6):383-95.

3. Baltzer A W, Lattermann C, Whalen J D, et al. Genetic enhancement of fracture repair: healing of an experimental segmental defect by adenoviral transfer of the BMP-2 gene. Gene Ther 2000;7(9):734-9.

4. Blagosklonny M V, el-Deiry W S. In vitro evaluation of a p53-expressing adenovirus as an anti-cancer drug. Int J Cancer 1996;67(3):386-92.

5. Brandsma J L, Brownstein D G, Xiao W, Longley B J. Papilloma formation in human foreskin xenografts after inoculation of human papillomavirus type 16 DNA. J Virol 1995;69(4):2716-21.

6. Carmeliet P, Stassen J M, Van Vlaenderen I, Meidell R S, Collen D, Gerard R D. Adenovirus-mediated transfer of tissue-type plasminogen activator augments thrombolysis in tissue-type plasminogen activator-deficient and plasminogen activator inhibitor-1-overexpressing mice. Blood 1997;90(4):1527-34.

7. Carmeliet P, Moons L, Lijnen R, et al. Inhibitory role of plasminogen activator inhibitor-1 in arterial wound healing and neointima formation: a gene targeting and gene transfer study in mice [see comments]. Circulation 1997;96(9):3180-91.

8. de Martin R, Raidl M, Hofer E, Binder B R. Adenovirus-mediated expression of green fluorescent protein. Gene Ther 1997;4(5):493-5.

9. Descamps V. [Skin and gene therapy. Hopes and realities]. Ann Dermatol Venereol 1995;122(4):226-34.

10. Fenjves E S. Approaches to gene transfer in keratinocytes. J Invest Dermatol 1994;103(5 Suppl):70S-75S.

11. Goebel E A, Davidson B L, Graham S M, Kern J A. Tumor reduction in vivo after adenoviral mediated gene transfer of the herpes simplex virus thymidine kinase gene and ganciclovir treatment in human head and neck squamous cell carcinoma. Otolaryngol Head Neck Surg 1998;119(4):331-6.

12. Goldhaber S Z. Thrombolytic therapy. Adv Intern Med 1999;44:311-25.

13. Graham F L, Eb AJvd. A new technique for the assay of infectivity of human adenovirus 5 DNA. Virology 1973;52(2):456-67.

14. Harvey B G, Worgall S, Ely S, Leopold P L, Crystal R G. Cellular immune responses of healthy individuals to intradermal administration of an E1-E3- adenovirus gene transfer vector. Hum Gene Ther 1999;10(17):2823-37.

15. Hsu M Y, Shih D T, Meier F E, et al. Adenoviral gene transfer of beta3 integrin subunit induces conversion from radial to vertical growth phase in primary human melanoma [see comments]. Am J Pathol 1998;153(5):1435-42.

16. Hsu M, Andl T, Li G, Meinkotk J L, Herlyn M. Cadherin repertoire determines partner-specific gap junctional communication during melanoma progression. J Cell Sci 2000;113(Pt 9):1535-42.

17. Hsu M Y, Meier F E, Nesbit M, et al. E-cadherin expression in melanoma cells restores keratinocyte-mediated growth control and down-regulates expression of invasion-related adhesion receptors. Am J Pathol 2000;156(5):1515-25.

18. Huber M, Limat A, Wagner E, Holil D. Efficient in vitro transfection of human keratinocytes with an adenovirus-enhanced receptor-mediated system. J Invest Dermatol 2000; 114(4):661-6.

19. Juhasz I, Albelda S M, Elder D E, et al. Growth and invasion of human melanomas in human skin grafted to immunodeficient mice. Am J Pathol 1993; 143(2):528-37.

20. Leconte I, Fox J C, Baldwin H S, Buck C A, Swain J L. Adenoviral-mediated expression of antisense RNA to fibroblast growth factors disrupts murine vascular development. Dev Dyn 1998;213(4):421-30.

21. Lee C T, Wu S, Gabrilovich D, et al. Antitumor effects of an adenovirus expressing antisense insulin-like growth factor I receptor on human lung cancer cell lines. Cancer Res 1996;56(13):3038-41.

22. Lee W C, Zhong C, Qian S, et al. Phenotype, function, and in vivo migration and survival of allogeneic dendritic cell progenitors genetically engineered to express TGF-beta. Transplantation 1998;66(12):1810-7.

23. Liechty K W, Nesbit M, Herlyn M, Radu A, Adzick N S, Crombleholme T M. Adenoviral-mediated overexpression of platelet-derived growth factor-B corrects ischemic impaired wound healing. J Invest Dermatol 1999;113(3):375-83.

24. Lu B, Scott G, Goldsmith L A. A model for keratinocyte gene therapy: preclinical and therapeutic considerations. Proc Assoc Am Physicians 1996;108(2):165-72.

25. Lu B. Federoff H J, Wang Y, Goldsmith L A, Scott G. Topical application of viral vectors for epidermal gene transfer. J Invest Dermatol 1997;108(5):803-8.

26. Meschia J F. Management of acute ischemic stroke. What is the role of tPA and antithrombotic agents? Postgrad Med 2000;107(6):85-6, 89-93.

27. Mohan P M, Chintala S K, Mohanam S, et al. Adenovirus-mediated delivery of antisense gene to urokinase-type plasminogen activator receptor suppresses glioma invasion and tumor growth. Cancer Res 1999;59(14):3369-73.

28. Morris J C, Ramsey W J, Wildner O, Muslow H A, Aguilar-Cordova E, Blaese R M. A phase I study of intralesional administration of an adenovirus vector expressing the HSV-1 thymidine kinase gene (AdV.RSV-TK) in combination with escalating doses of ganciclovir in patients with cutaneous metastatic malignant melanoma. Hum Gene Ther 2000;1 l(3):487-503.

29. Nagai N, De Mol M, Lijnen H R, Carmeliet P, Collen D. Role of plasminogen system components in focal cerebral ischemic infarction: a gene targeting and gene transfer study in mice. Circulation 1999;99(18):2440-4.

30. Nesbit M, Nesbit H K, Bennett J, et al. Basic fibroblast growth factor induces a transformed phenotype in normal human melanocytes. Oncogene 1999;18(47):6469-76.

31. Nesbit M, Helmut Schaider, Thomas Andl, Daw-Tsun Shih, Mei-Yu Hsu, Michelle McBrian, Timothy M. Crombleholme, Carola Berking, Rosalie Elenitsas, Meenhard Herlyn. a5-Integrin Gene Transfer Mimics PDGF-B Transformed Phenotypes in Human Dermal Fibroblasts and Human Skin (in press) 2000.

32. Ohsawa T, Nakamura T, Mihara M, Sato K. Enhancement of adenovirus-mediated gene transfer into dermal fibroblasts in vitro and in vivo by polyethylene glycol 6000. J Dermatol 2000;27(4):244-51.

33. Oka M, Berking C, Nesbit M, et al. Interleukin-8 overexpression is present in pyoderma gangrenosum ulcers and leads to ulcer formation in human skin xenografts. Lab Invest 2000;80(4):595-604.

34. Phaneuf D, Chen S J, Wilson J M. Intravenous injection of an adenovirus encoding hepatocyte growth factor results in liver growth and has a protective effect against apoptosis. Mol Med 2000;6(2):96-103.

35. Prabhu N S, Blagosklonny M V, Zeng Y X, Wu G S, Waldman T, El-Deiry W S. Suppression of cancer cell growth by adenovirus expressing p21 (WAF1/C1P1) deficient in PCNA interaction. Clin Cancer Res 1996;2(7):1221-9.

36. Satyamoorthy K, Oka M., Herlyn M. An Antisense Strategy for Inhibition of Human Melanoma Growth Targets the Growth Factor Pleiotrophin. Pigment Cell Res 2000; In press.

37. Sauter E R, Nesbit M, Watson J C, Klein-Szanto A, Litwin S, Herlyn M. Vascular endothelial growth factor is a marker of tumor invasion and metastasis in squamous cell carcinomas of the head and neck. Clin Cancer Res 1999;5(4):775-82.

38. Sauter E R, Nesbit M, Litwin S, Klein-Szanto A J, Cheffetz S, Herlyn M. Antisense cyclin D1 induces apoptosis and tumor shrinkage in human squamous carcinomas. Cancer Res 1999;59(19):4876-81.

39. Setoguchi Y, Jaffe H A, Danel C, Crystal R G. Ex vivo and in vivo gene transfer to the skin using replication-deficient recombinant adenovirus vectors. J Invest Dermatol 1994;102(4):415-21.

40. Sexton C J, Williams A T, Topley P, et al. Development and characterization of a novel xenograft model permissive for human papillomavirus DNA amplification and late gene expression. J Gen Virol 1995;76(Pt 12):3107-12.

41. Steele T A. Recent developments in the virus therapy of cancer. Proc Soc Exp Biol Med 2000;223(2):118-27.

42. Sterman D H, Treat J, Litzky L A, et al. Adenovirus-mediated herpes simplex virus thymidine kinase/ganciclovir gene therapy in patients with localized malignancy: results of a phase I clinical trial in malignant mesothelioma. Hum Gene Ther 1998;9(7):1083-92.

43. Stewart A K, Lassam N J, Quirt I C, et al. Adenovector-mediated gene delivery of interleukin-2 in metastatic breast cancer and melanoma: results of a phase 1 clinical trial. Gene Ther 1999; 6(3):350-63.

44. Sylvester K G, Nesbit M, Radu A, Herlyn M, Adzick N S, Crombleholme T M. Adenoviral-mediated gene transfer in wound healing: acute inflammatory response in human skin in the SCID mouse model. Wound Repair Regen 2000;8(1):36-44.

45. Thurston G, Rudge J S, Ioffe E, et al. Angiopoietin-1 protects the adult vasculature against plasma leakage. Nat Med 2000;6(4):460-3.

46. Tumova S, Woods A, Couchman J R. Heparan sulfate proteoglycans on the cell surface: versatile coordinators of cellular functions. Int J Biochem Cell Biol 2000;32(3):269-88.

47. Veelken H, Jesuiter H, Mackensen A, et al. Primary fibroblasts from human adults as target cells for ex vivo transfection and gene therapy. Hum Gene Ther 1994;5(10):1203-10.

48. Vlodavsky I, Miao H Q, Medalion B, Danagher P, Ron D. Involvement of heparan sulfate and related molecules in sequestration and growth promoting activity of fibroblast growth factor. Cancer Metastasis Rev 1996;15(2):177-86.

49. Yan H C, Juhasz I, Pilewski J, Murphy G F, Herlyn M, Albelda S M. Human/severe combined immunodeficient mouse chimeras. An experimental in vivo model system to study the regulation of human endothelial cell-leukocyte adhesion molecules. J Clin Invest 1993;91(3):986-96.

We claim:

1. A method of vascularizing an autologous human skin graft, comprising the step of:
injecting the skin graft intradermally with a vector comprising a polynucleotide encoding a polypeptide selected from the group consisting of tPA, MEL-CAM, uPA, SCF, bFGF, IGF-1, and ET-3, whereby the skin graft becomes vascularized.

2. The method of claim 1, wherein the vector encodes at least two different polypeptides.

3. The method of claim 1, wherein the skin graft is injected with at least two vectors, each vector encoding a different polypeptide.

4. The method of claim 1, wherein the vector encodes bFGFs.

5. The method of claim 1, wherein bone marrow derived stem cells are recruited to the skin graft to form fibroblasts and endothelial cells.

6. The method of claim 1 comprising the further step of:
implanting bone marrow derived stem cells into the skin graft.

7. The method of claim 6 wherein the stem cells are autologous.

8. The method of claim 1, wherein the skin graft is injected at least three times with the vector.

9. The method of claim 1, wherein the skin graft is injected with the vector prior to being transplanted.

10. The method of claim 1, wherein the skin graft is injected with the vector after being transplanted.

11. The method of claim 1, wherein the vector is a replication-deficient adenoviral vector.

12. The method of claim 1, wherein the vector is a replication-deficient retroviral vector.

13. A skin graft system comprising an immunodeficient mouse having a human skin graft which has been injected intradermally with a vector comprising a polynucleotide encoding a polypeptide selected from the group consisting of tPA, MEL-CAM, uPA, SCF, bFGF, IGF-1, and ET-3.

14. The skin graft system of claim 13, wherein the mouse is a SCID mouse.

15. The skin graft system of claim 13, wherein the skin graft comprises cancer cells.

16. The skin graft system of claim 15, wherein the cancer cells are melanoma cells.

17. The skin graft system of claim 13, wherein the skin graft comprises bone marrow derived stem cells.

18. The skin graft system of claim 17, wherein the stem cells are implanted into the skin graft.

19. A method of preparing the skin graft system of claim 13, comprising the step of:
injecting intradermally a human skin graft which has been transplanted onto an immunodeficient mouse with a vector comprising a polynucleotide encoding a polypeptide selected from the group consisting of tPA, MEL-CAM, uPA, SCF, bFGF, IGF-1, and ET-3.

20. The method of claim 19, wherein the mouse is a SCID mouse.

21. A vascularized human skin graft made by the method of claim 19, wherein the skin graft has become vascularized.

22. A method of identifying a therapeutic drug candidate for treatment of melanoma, comprising the step of:
determining melanoma cell proliferation, dispersal, or survival in a skin graft of claim 16 in the presence of a test substance, wherein the test substance is identified as a therapeutic drug candidate if it inhibits melanoma cell proliferation, dispersal, or survival.

23. The method of claim 22, wherein the test substance is an antisense oligonucleotide comprising 15 or more consecutive nucleotides of the complement of a polynucleotide sequence encoding TGF-β1, collagen type VI, collagen type XV, collagen type XVIII, tenascin, PAI-I, VEGF, CFR-1, PDGF receptor β, or HGF.

24. The method of claim 22, wherein the test substance is an antibody that specifically binds a polypeptide selected from the group consisting of TGF-β1, collagen type VI, collagen type XV, collagen type XVIII, tenascin, PAI-I, VEGF, CFR-1, PDGF receptor β, and HGF.

25. The method of claim 22, wherein the skin graft has been injected intradermally with a vector comprising a polynucleotide encoding E-cadherin or desmoglein-1.

26. A method of identifying a therapeutic drug candidate for inducing vascularization in a skin graft, comprising the step of:
determining vascularization of a skin graft of claim 13 in the presence of a test substance, wherein the test substance is identified as a therapeutic drug candidate if it induces vascularization.

27. A method of identifying a therapeutic drug candidate for treatment of wounds, comprising the step of:
wounding a skin graft of claim 13 in the presence of a test substance; and
determining the rate or degree of wound healing, wherein the test substance is identified as a therapeutic candidate if it increases the rate or degree of wound healing.

28. A method of inducing vascularization in a mammalian tissue, comprising the step of:
injecting into the tissue a vector comprising a polynucleotide encoding a polypeptide selected from the group consisting of tPA, MEL-CAM, uPA, SCF, bFGF, IGF-1, and ET-3.

29. The method of claim 28, wherein the vector encodes at least two different polypeptides are encoded.

30. The method of claim 28, wherein bone marrow derived mesenchymal stem cells are recruited to the tissue.

31. The method of claim 28 comprising the further step of:
implanting bone marrow derived stem cells into the tissue.

32. The method of claim 28, wherein the tissue is heart muscle.

33. The method of claim 28, wherein the vector is a replication-deficient adenoviral vector.

34. The method of claim 28, wherein the tissue is injected at least three times.

35. The method of claim 28, wherein the mammal has diabetes.

36. The method of claim 28, wherein the tissue is at the site of a burn.

37. The method of claim 28, wherein the tissue is at the site of a wound.

38. The method of claim 37, wherein the wound is a venous leg ulcer.

39. An artificial microvascularized dermal reconstruct comprising:
a monolayer of human endothelial cells;
a matrix comprising a first layer which is adjacent to the monolayer of endothelial cells and a second layer which is adjacent to the first layer, wherein the first layer comprises collagen type I and the second layer comprises fibroblasts and collagen type I; and
interconnected microvascular spaces within the matrix.

40. The dermal reconstruct of claim 39, wherein the second layer further comprises smooth muscle cells.

41. The dermal reconstruct of claim 39 or 40, wherein the second layer further comprises bone marrow derived stem cells.

42. The dermal reconstruct of claim 39, wherein at least one cell type selected from the group consisting of said fibroblasts and said endothelial cells has been transduced with a vector which expresses a polypeptide selected from the group consisting of VEGF, Ang 1, Ang 2, tPA, uPA, VEGF, MEL-CAM, SCF, PDGF-A, VEGF-D, TGF-β1, VEGF-C, PDGF-D, bFGF, IGF-1, and ET-3.

43. The dermal reconstruct of claim 42, wherein at least one cell type selected from the group consisting of said fibroblasts and said endothelial cells expresses a polypeptide selected from the group consisting of tPA, MEL-CAM, uPA, SCF, bFGF, IGF-1, and ET-3.

44. The dermal reconstruct of claim 40, wherein at least one cell type selected from the group consisting of said fibroblasts, said endothelial cells, and said smooth muscle cells has been transduced with a vector which expresses a polypeptide selected from the group consisting of VEGF, Ang 1, Ang 2, tPA, uPA, VEGF, MEL-CAM, SCF, PDGF-A, VEGF-D, TGF-b1, VEGF-C, PDGF-B, bFGF, IGF-1, and ET-3.

45. The dermal reconstruct of claim 44, wherein at least one cell type selected from the group consisting of said fibroblasts, said endothelial cells, and said smooth muscle cells expresses a polypeptide selected from the group consisting of tPA, MEL-CAM, uPA, SCF, bFGF, IGF-1, and ET-3.

46. The dermal reconstruct of claim 42 or 44, wherein at least two polypeptides selected from the group consisting of VEGF, Ang 1, Ang 2, tPA, uPA, VEGF, MEL-CAM, SCF, PDGF-A, VEGF-D, TGF-b1, VEGF-C, PDGF-B, bFGF, IGF-1, and ET-3 are expressed.

47. The dermal reconstruct of claim 42, 43, 44, 45, or 46, wherein the vector is a replication deficient adenoviral vector.

48. The dermal reconstruct of claim 42, 43, 44, 45, or 46, wherein the vector is a replication deficient retroviral vector.

49. A method for producing a dermal reconstruct in vitro, comprising the steps of:
overlaying a monolayer of endothelial cells with a first layer comprising collagen type I; and
overlaying the first layer with a second layer comprising both collagen type I and fibroblasts to form a dermal reconstruct, wherein at least one cell type of said endothelial cells and said fibroblasts has been transduced using a vector comprising a polynucleotide encoding a polypeptide selected from the group consisting of VEGF, Ang 1, Ang 2, tPA, uPA, MEL-CAM, SCF, PDGF-A, VEGF-D, TGF-b1, VEGF-C, PDGF-B, bFGF, IGF-1 and ET-3.

50. The method of claim 49 further comprising the step of:
growing the dermal reconstruct in culture until it becomes vascularized.

51. The method of claim 49, wherein the second layer further comprises bone marrow derived stem cells.

52. The method of claim 49 wherein VEGF, Ang 1, Ang 2, or tPA is encoded.

53. The method of claim 52 wherein cells of the dermal reconstruct have been transduced with polynucleotides encoding both VEGF and Ang 1.

54. The method of claim 49, wherein the vector is a replication deficient adenoviral vector.

55. The method of claim 49, wherein the vector is a replication deficient retroviral vector.

56. A method of treating a mammal having a condition selected from the group consisting of a wound, a burn, and an ischemic tissue, the method comprising the step of:
administering to the mammal at the site of the condition a vascularized dermal reconstruct made by the method of claim 50.

57. The method of claim 56, wherein the mammal is diabetic.
